(19) United States
(12) Patent Application Publication
(10) Pub. No.: US 2008/0086104 A1
(43) Pub. Date: Apr. 10, 2008

(54) Absorbent article having improved fastening means

(75) Inventor: Matilda Karlsson, Goteborg (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 11/987,381

(22) Filed: Nov. 29, 2007

Related U.S. Application Data
(63) Continuation of application No. PCT/SE2005/000881, filed on Jun. 13, 2005.

(51) Int. Cl. A61F 13/64

(52) U.S. Cl. 604/385.03

(57) ABSTRACT

The invention concerns an absorbent article, such as a diaper, pant diaper, incontinence guard, panty liner, open diaper, belt diaper or the like. More specifically, the invention concerns a fastening device for an absorbent article having a receiving portion with an opening, and a tab element which is shaped so that it can be inserted into the opening of the receiving portion upon closing the article. It is characteristic of the invention that both the tab element and the receiving portion have fastening means. By the fact that both the receiving portion and the tab element have fastening means, a strong and stable connection is formed between the components of the fastening device.

### TECHNICAL FIELD

The present invention concerns an absorbent article, such as a diaper, pant diaper, incontinence guard, panty liner, open diaper, belt diaper and the like. Specifically, the invention concerns a fastening device for an absorbent article having a receiving portion with an opening, and a tab element which is shaped so that it can be inserted into the opening of the receiving portion to close the article.

### BACKGROUND

GB-A-2,303,169 describes how a fastening system with openings is used for an absorbent article. The fastening system has a first and a second fastening element, which are intended to hook fast in each other after the article has been applied to a user. In order to strengthen and obtain good fastening, the first fastening element comprises an elastic structure with a plurality of openings. The structure is configured so that the openings expand when the structure is stretched and close when the elastic structure contracts. The second fastening element has hook material, the hooks of which—upon application—fasten in the opening which is formed when the elastic material is stretched. The solution is not particularly practical, as it provides a weak close and is difficult to apply so that all hooks meet with an opening.

Another previously-known fastening device for an absorbent article is described in EP 1,009,350 B1. The fastening device has a receiving portion with an opening for insertion of a fastening portion. The fastening portion does not fasten directly to the receiving portion, but rather “hooks” fast in the opening via the special shape of the fastening portion. The fastening device is said to be strong and simple. Another advantage which is given is that the fastening device has a designated fastening configuration.

More specifically, this means that the fastening device has a receiving portion which comprises an inner portion and an outer portion as well as an opening placed therebetween. The inner portion is joined to the absorbent article. The fastening portion has a proximal portion joined to the absorbent article and a distal portion. The distal portion has something which is comparable with a tongue portion which is formed by folding of the distal portion of the fastening portion. The fastening portion with tongue portion is inserted through the opening in the receiving portion so that at least the tongue portion extends over the outer portion of the receiving portion and, in use, thereby locks the fastening portion tightly to the receiving portion.

With the so-called “designated fastening configuration”, no extra material is required to ensure good fastening of the article. A further advantage is said to be that the article can be fastened together without the side panels overlapping, which is said to save material and thereby reduce the manufacturing costs of the article.

There are, however, a series of disadvantages with the above-described fastening device. While the opening itself is meant to function as a fastening means to the fastening portion which runs through the opening, the fastening device is dependent upon the shape of the fastening portion and the opening being harmonized. A fastening device of the above-mentioned kind therefore requires that a force holds the device in a tensioned state. In the event that the user draws in their stomach, the device can temporarily slacken, and thereby risk loosening. There is also a certain play in such a fastening device, where the fastening portion only hooks into the receiving portion. Moreover, at high stresses on the fastening device, the fastening portion with the tongue portion can collapse, i.e. fold together, and thereby be pulled out of the opening.

### SUMMARY OF THE INVENTION

In accordance with the present invention, an absorbent article such as a diaper, pant diaper, sanitary napkin, incontinence guard, panty liner, open diaper, belt diaper, bed protector and the like has been provided which substantially avoids or minimizes the problems of known articles. An article according to the invention is primarily characterized by both the tab portion and the receiving portion having fastening means.

Through both the tab portion and the receiving portion having fastening means, a stable and secure seal is obtained. The tab portion which is inserted into the opening of the receiving portion can be fastened simply to the receiving portion and/or to the article, and thereby strengthen the fastening. The fastening means on the receiving portion provides further stabilization of the fastening device. Strengthening not only provides a more secure fastening, but also a better fit, as the play in the fastening device is eliminated. In one embodiment, the fastening means may be formed from hook material, which gives a firm and stable connection and is a simple and low-cost construction solution when compared to the prior art. In one embodiment, the tab portion and the receiving portion have fastening means for fastening to the tab portion. This can be advantageous when one requires a long receiving portion and a long tab element as the fastening device, e.g. for a belt diaper. In another embodiment, the tab portion and/or the receiving portion have fastening means for fastening to the absorbent article. Such a configuration stabilizes the connection between the side panels of the article so that play—and thereby leakage—is minimized.

To provide good fit, it is important that there is no play in the fastening device. This can be minimized by a tab element and receiving portion having fastening means, preferably in the form of hook material. Together with the opening in the receiving portion, a stable connection is provided, which in practice should be able to replace a weld in the side-seams of a pant diaper. A further strengthening of the seal is provided if the receiving portion has at least two separate hook materials placed adjacent to the opening. By placing the hook material at different positions around the opening, different functions are obtained. For example, the distal portion can be used as a grip-tab when at least two separate hook materials are placed between the proximal portion and the distal portion. This can be advantageous if the product is to be aimed at users requiring strong, yet grippable, fastening devices. In such a configuration, the two separate hook materials preferably extend at least along half the length of the opening.

In a simple embodiment, the tab element and the receiving portion can comprise at least one layer of nonwoven. The tab element and the receiving portion are preferably a laminate of at least two layers. The laminate can for example be formed from 20 g/m² spunbond and a 20 g/m² carded nonwoven layer. Both layers are preferably made of polypropene fibres. The hook material can for example be a VELCRO® material. The tab element and the receiving portion are preferably elastic, and can be formed for example of an elastic laminate comprising an elastic film laminated with a nonwoven material. Further examples of elastic laminates are described in EP 1,261,479 B 1, US 20020016122 A1 and EP 757,624 B1. The tab element and the receiving portion can also exhibit elastic zones. Examples of elastic zones can be the proximal portion of the tab element, the proximal portion of the receiving portion, the distal portion of the tab element or the distal portion of the receiving portion. The tab element and the receiving portion can also exhibit further elastic zones, for example an elastic zone in the proximal portion and also one in the distal portion. The zones can be separated by a non-elastic zone. The elasticity can also be different for the different zones. In yet another embodiment, either the tab element or the receiving portion are elastic.

The fastening means can fasten directly to the receiving portion, tab element or to the absorbent article. The fastening means can also fasten to a loop material, or a special surface intended for a specific fastening means. When the fastening means is glue, the special surface may be a silicone-treated surface in order to facilitate fastening.

In one embodiment, the distal portion of the tab element can have a similar form to the opening of the receiving portion. Preferably, the distal portion of the tab element has a pointed shape, which indicates visually to the user that it is this portion which should be inserted through the opening. The opening of the receiving portion can also have a similar pointed shape, for example such as that shown in FIG. 5b. The point on the opening of the receiving portion, together with the pointed shape of the tab element indicates visually to the user how the fastening device should be arranged.

When the fastening device according to the invention is applied on a light incontinence guard, a sanitary napkin or panty liner, it is preferably placed on the “wings”. As an alternative, the fastening device can form part of the wings. The product can then be fastened to underwear with the help of the fastening device, so as to avoid the product sliding out of place in use.

### BRIEF DESCRIPTION OF THE FIGURES

These, and other objects, features, and advantages of the present invention will become more readily apparent to those skilled in the art upon reading the following detailed description, in conjunction with the appended drawings in which:

FIG. 1 shows a diaper in an outstretched state seen towards the liquid permeable topsheet.

FIG. 2 shows a diaper in an assembled state.

FIGS. 3a-3c show different configurations of the fastening means on the fastening device.

FIGS. 4a-4b show different dimension specifications for the receiving portion and the tab element.

FIGS. 5a-5d show different forms of the opening in the receiving portion.

FIG. 6 shows a different configuration of the receiving portion and tab element.

FIG. 7 shows a further different configuration of the receiving portion and tab element.

### DETAILED DESCRIPTION OF THE PREFERRED EMBODIMENTS

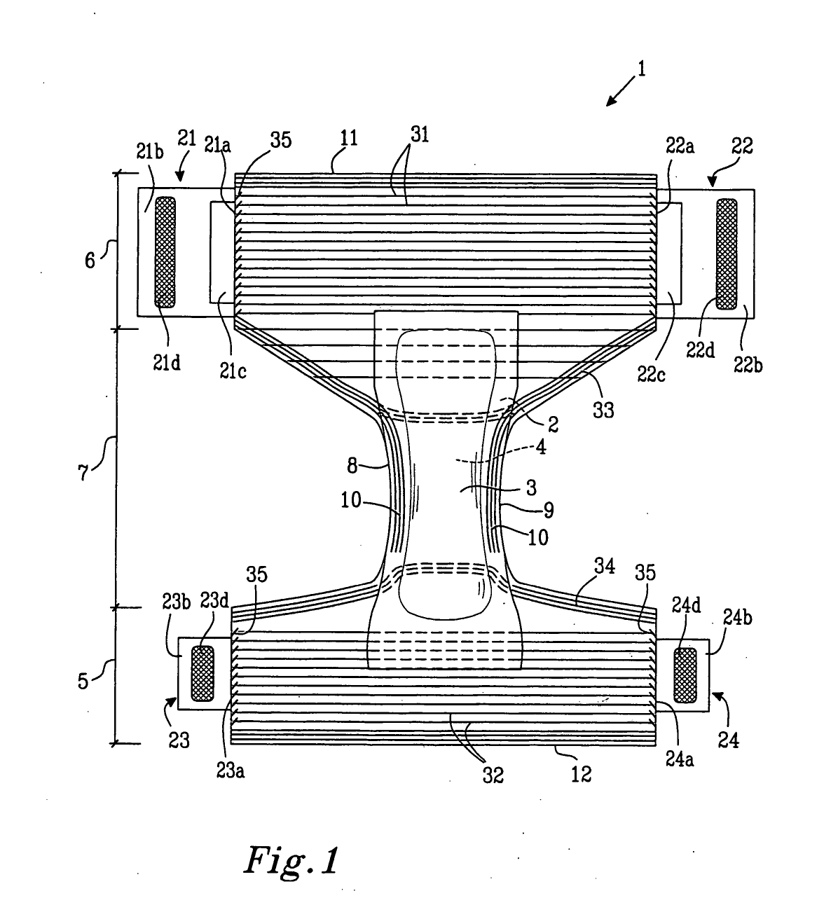

FIG. 1 shows an embodiment of an absorbent article in the form of a pant diaper 1 comprising a backsheet material 2 which is suitably liquid-impermeable, a liquid-permeable topsheet 3 and an absorbent body 4 contained therebetween. The liquid permeable topsheet 3 may comprise nonwoven material, e.g. spunbond material of continuous filaments, melt-blown material, bonded carded fibre material, perforated plastic film or combinations thereof. In the case where the backsheet material 2 is liquid-impermeable, it may comprise material such as a plastic film, non-woven material which is coated with a liquid-repellent material or hydrophobic material which resists liquid penetration. The backsheet material 2 is preferably breathable. By “breathable” material, is meant a material which has the ability to allow water vapor and other gases through. Examples of such materials are well-known within the field and will not be described further.

The backsheet material 2 and the topsheet 3 have a somewhat greater extension in the plane than the absorbent body 4, and extend outside the edges of the absorbent body. The backsheet material 2 and the topsheet 3 are connected to each other within the extended regions, e.g. via gluing or welding with heat or ultrasound. Elastic elements 10 run along each side of, and substantially parallel with, the absorbent body 4. So-called “raised leakage barriers” (not shown) may also be positioned in the longitudinal direction by the absorbent body 4. The article has two longitudinal edges 8,9 as two transverse edges 11,12.

The absorbent body 4 can be of any conventional type. Examples of commonly found absorbent materials are cellulose fluff pulp, tissue layers, superabsorbent polymers (so called “superabsorbents” or SAP), absorbent foam material, absorbent nonwoven material and the like. It is common to combine cellulose fluff pulp with superabsorbents in an absorbent body. It is also common to build up absorbent bodies with layers of different materials with different properties regarding liquid uptake properties, distribution properties and storage properties. This is well-known to a person skilled in the art and therefore need not be described in detail. The thin absorbent cores which are common in e.g. baby diapers and incontinence guards often comprise a compressed, mixed or layered structure of cellulose fluff pulp and superabsorbents. Examples of absorbent cores which should be suitable for the absorbent article are described in detail in published patents EP 659,541 and WO 93/21882. However, the invention is not limited to just these, but these should rather be seen as examples of absorbent cores.

The diaper shown in FIG. 1 can for example be produced by joining the absorbent core to a backsheet material as a finished core packet. The core packet thus comprises a liquid-impermeable backsheet, an absorbent core and a topsheet which are ready-laminated for application on a carrier nonwoven.

The pant diaper 1 is intended to enclose the lower part of the wearer's trunk like a pair of absorbent pants. It has a front part 5 intended in use to face forwards on the user, and a rear part 6 intended in use to face rearwards on the user. A crotch part 7 is located between the front and rear parts, which is intended to be placed in the user's crotch between their legs. The pant diaper has a longitudinal direction and a transverse direction. The front part and the rear part of the diaper have elastic elements 31, 32. In the Figures, the elastic elements 31, 32 are elastic threads which extend in the transverse direction. The diaper also has leg elastic 33, 34 which extends between the longitudinal edges 8, 9 of the article, directly over the absorbent core 4 in the crotch region 7. The leg elastic 33, 34 runs partly along the longitudinal edges 8, 9 of the article, so that leg elastic is formed around the entire longitudinal edges 8, 9 in the crotch region 7, together with the elastic elements 10. The entire, or part of the elastic 31, 32, 33, 34 is placed between the backsheet 2 and absorbent core 4 and has been drawn with dashed lines for the sake of illustration.

The pant diaper 1 has a fastening device 21, 22, 23, 24 formed from two receiving portions 21, 22 placed in the rear part 6 of the diaper 1, and two tab elements 23, 24 placed in the front part 5 of the diaper 1. The two receiving portions 21, 22 each have a proximal portion, 21a, 22a and a distal portion 21b, 22b and an opening 21c, 22c located therebetween. In the Figure shown, the proximal portion 21a, 22a of the receiving portions 21, 22 is comprised partly of the longitudinal side edge 8, 9 in the back part 6. The two tab elements 23, 24 of the diaper 1 have a proximal portion 23a, 24a joined to the longitudinal edges 8, 9 of the diaper, and a distal portion 23b, 24b. The distal portion 23b, 24b is preferably shaped so that it can be inserted into the opening 21c, 22c of the receiving portion 21, 22 to close the article 1.

The fastening device 21, 22, 23, 24 also has fastening means 21d, 22d, 23d, 24d in the form of hook material. The hook material 21d, 22d on the receiving portion 21, 22 of the article 1 extends in the longitudinal direction of the article 1, along the distal portion 21b, 22b of the receiving portion 21, 22. In the embodiment shown, the hook material 21d, 22d extends substantially the same distance in the longitudinal direction as the opening 21c, 22c. This ensures a good fit, at the same time as a strong and secure connection is obtained. In one embodiment, the pant diaper can be permanently closed along the longitudinal edges of the article 1 in the front part 5 and the rear part 6 by ultrasonic welding, indicated in FIG. 1 with weld-lines 35, so that the article 1 forms a reclosable pant diaper. When the permanent seals are torn and broken, the article can be closed again with the help of the fastening device 21, 22, 23, 34. In that the longitudinal edges 8, 9 in the rear part 6 form a part of the opening 21c, 22c of the receiving portion 21, 22, the advantage is obtained that the diaper does not change its fit when the diaper is closed again with the fastening device 21, 22, 23, 24. When the diaper 1 is closed again with the help of the fastening device 21, 22, 23, 24, the longitudinal edges 8, 9 of the diaper 1 end up in essentially the same position as before the permanent seal is broken.

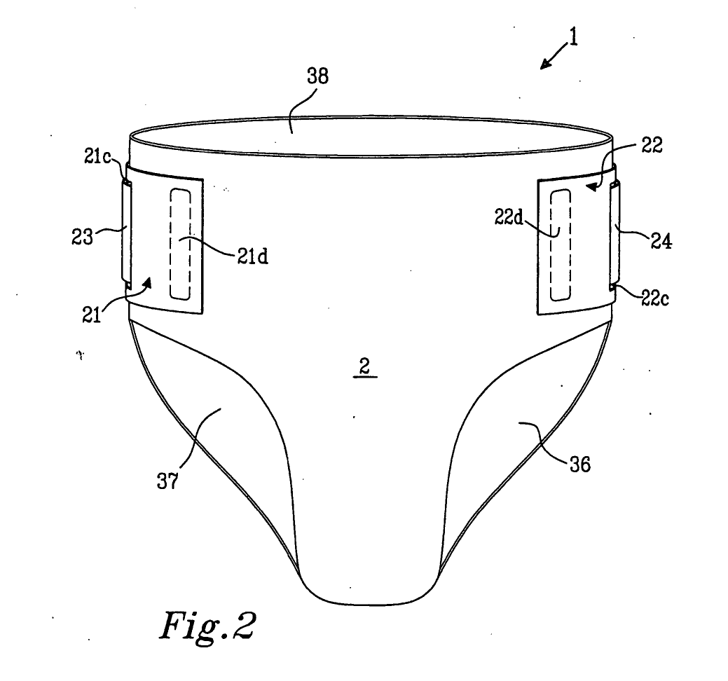

In FIG. 2, the pant diaper 1 is shown in its assembled state. The tab elements 23, 24 of the diaper have been inserted through the opening 21c, 22c of the receiving portion 21, 22. Hook material 2ld, 22d is joined to the backsheet 2 on the front part 5 of the diaper and the hook material 23d, 24d of the tab elements 23, 24 is joined in connection to the back part 6 of the diaper. FIG. 2 also shows a waist opening 38 and two leg openings 36, 37.

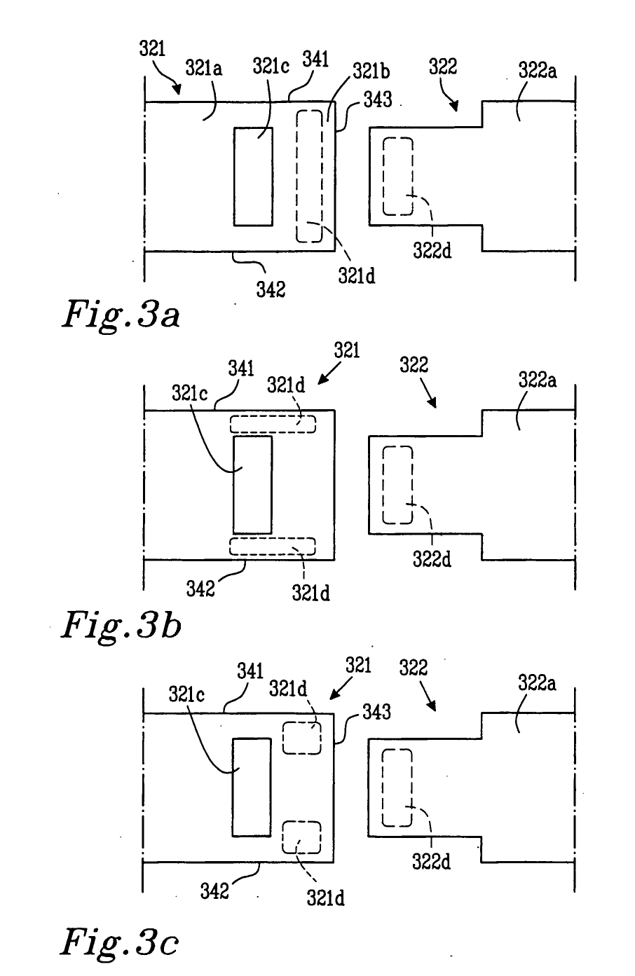

FIGS. 3a-c show fastening devices with different fastening means which can be configured in various ways to an absorbent article. More precisely, FIG. 3a shows a receiving portion 321 having a proximal portion 321a and a distal portion 321b and an opening 321c located therebetween. The proximal portion 321a can be joined to an absorbent article so as to partly form the receiving portion of a fastening device. The receiving portion has two longitudinal edges 341, 342 and a transverse edge 343 in connection with the distal end 321b of the receiving portion.

Depending on how the proximal portion 321a joins to the absorbent article, the way in which the receiving portion 321 defines the entire opening 321c or whether the absorbent article should form a part of the opening 321c as shown in FIG. 1 may be chosen. FIG. 3a also shows a tab element 322 having a proximal portion 322a and a distal portion 322b. The proximal portion 322a can be joined to an absorbent article in order to partly form a fastening device. The distal portion 322b is formed so that it can be inserted into the opening 321c of the receiving portion 321, for closure. Depending on which side the distal portion 322b of the tab element 322 is inserted into the opening from, it may be attached to the receiving portion 321 or direct to the absorbent article. The extent to which the distal portion 322b of the tab element 322 is fastened to the receiving portion 321 or to the absorbent article depends on how one wishes the fastening device to function. For example, the distal portion 322b of the tab element 322 can be sufficiently long that it extends past the receiving portion 321 and thereby fastens to the absorbent article. Alternatively, the receiving portion 321 can join to the longitudinal edge of the absorbent article in such a way that it does not significantly overlap with the absorbent article. The absorbent article can also form part of the opening 321c on the receiving portion 321.

In another embodiment, the proximal portion on the receiving portion and/or on the tab element form part of the absorbent article. In a further embodiment, the receiving portion and/or tab element are formed of a separate piece of material.

The fastening means 321d, 322d can also be seen in FIG. 3a. The fastening means 321d, 322d may comprise glue, hook material or any other material known in the field. By the phrase “hook material” is hereby meant a material comprising a plurality of small hooks which can mechanically grip to another material—the receiving material. This receiving material can be a specially-formed loop material, or a fibre-based material, e.g. a non-woven. In this way, a non-woven material can serve as a receiving material for a hook material.

In FIG. 3a, the receiving portion 321 has a fastening means 321d in the form of hook material which extends along the transverse side 343 of the distal portion 321b. FIGS. 3b and 3c show further alternative placing of the fastening means 321d on the receiving portion 321. In FIG. 3b, two fastening means are placed on either side of the opening 321c along the longitudinal edges 341, 342 of the receiving portion.

In FIG. 3c, the fastening means 321d is placed in the corner between the longitudinal edge 341 and the transverse edge 343 of the receiving portion 321 and in the corner between the transverse edge 343 and the other longitudinal edge 342.

In one embodiment, the entire receiving portion 321 and/or tab element is formed of hook material. The opening 321c is then for example punched out of the hook material. In another embodiment of the invention, the tab element can be inserted through the opening in the receiving portion and fastened back on the tab element instead of being fastened to the receiving portion or to the absorbent article.

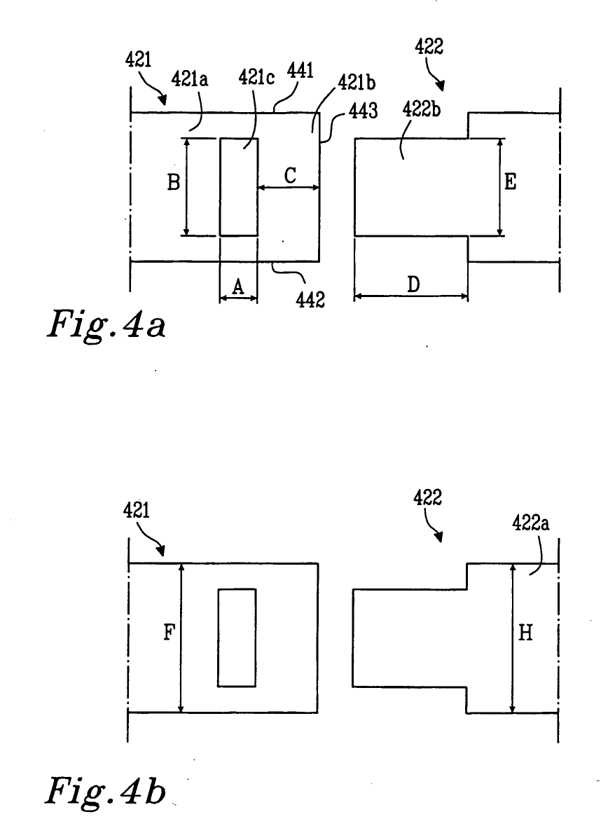

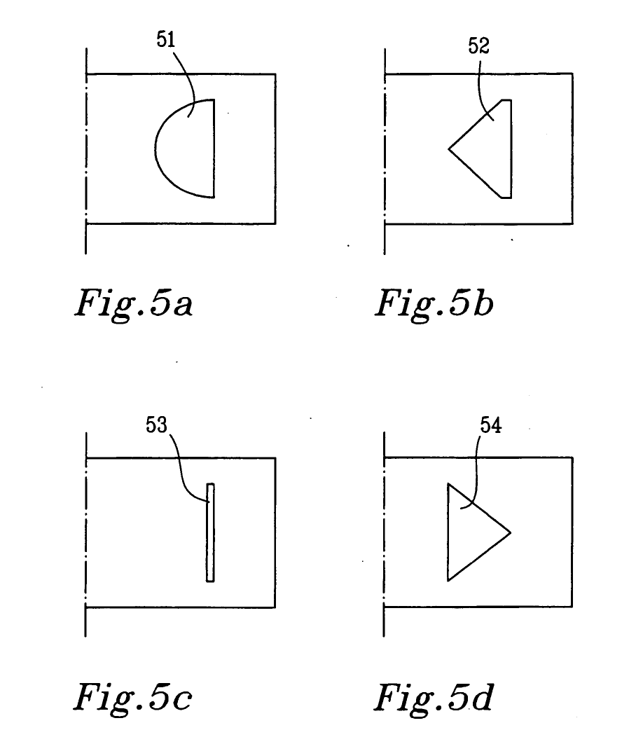

FIGS. 4a, 4b show an embodiment of a receiving portion 421 and a tab element 422 in schematic form to illustrate the various dimensions of a receiving portion, and a tab element. The receiving portion 421 has a first longitudinal edge 441, a second longitudinal edge 442 and a transverse edge 443 located therebetween. The receiving portion 421 has a proximal portion 421a and a distal portion 421b, and an opening 421c located therebetween. In the embodiment shown, the opening 421c has a rectangular form with a short edge A and a long edge B. It is considered within the scope of the invention that the length of sides A and B can be applied to other openings, for example those illustrated in FIGS. 5a-d, which can have a length A and a length B. Length A can be between 0.1-100 mm, 0.1-50 mm, 0.1-20 mm, 5-50 mm or 5-20 mm. A length of 0.1 mm means in practice that the opening is only a slit, as illustrated in FIG. 5c. The length B of the opening 421c can be between 10-200 mm, 10-100 mm, 10-80 mm, 50-100 mm or 30-80 mm. The distance between the opening 321c and the transverse edges is denoted in FIG. 4a as C. The distance C is suitably in the interval 1-50 mm, 1-10 mm, 10-50 mm or 10-30 mm.

The distal portion 422b of the tab element 422 extends a distance D in the longitudinal direction. The distance D is in the interval 10-200 mm, 10-100 mm, 30-150 mm, 30-100 mm, 40-100 mm or 20-80 mm. The width E of the distal portion is in the interval 10-200 mm, 10-100 mm, 10-80 mm 50-100 mm or 30-80 mm in the transverse direction. The proximal portions 421a, 422a of the receiving portion 421 and tab element 422 are preferably equally wide in the transverse direction. The receiving portion has a width F in the transverse direction.

The proximal portion 422a of the tab element 422 has a width H in the transverse direction. In certain embodiments, the width H of the proximal portion 422a may be the same as the width E of the distal portion 422b. This provides the opportunity for great variation in the size of the waist opening of the absorbent article. The article can thereby be fitted to a variety of different individuals of different sizes. If this is not the case, the difference between H and E is preferably equally large as the difference between distances F and B described above. Distance F and H are in the interval 100-20 mm, 100-50 mm or 50-20 mm

In one embodiment of the fastening device, the receiving portion is formed from a nonwoven laminate comprising a spunbond nonwoven layer of polypropylene fibres with a surface weight of 20 g/m², ultrasonically welded to a carded nonwoven layer of polypropylene fibres with a surface weight of 20 g/m². The bonding area is approximately 8% of the total area of the laminate.

The bonding pattern is a conventional one, e.g. checked pattern, circles, diamond patterns or the like. The hook material is of the VELCRO® type and is joined to the receiving portion and the tab element with a conventional hot melt adhesive. The configuration of the hook material is that illustrated in FIG. 3b. The area of two hook materials on the receiving portion is approximately 4 cm² each. The area of the hook material on the tab elements is approximately 7.5 cm².

The opening on the receiving portion is rectangular, with dimensions A=2 cm, B=4 cm. The distal portion is C=1.5 cm. The total length of the receiving portion is 8 cm. Other measurements are width F=7.5 cm. The measurements of the tab element are D=4 cm, E=3.9 cm, H=7.5 cm. The total length of the tab element is 8 cm. The tab element and receiving portion are firmly welded with ultrasound onto a pull-up type pants diaper so that a fastening device is obtained. The tab element and receiving portion could of course also be used on a conventional open type of diaper.

Further examples of shapes of the opening in the receiving portion 21, 22, 321, 421 are illustrated in FIG. 5a-5c. FIG. 5 shows a semicircular opening 51 for receiving the tab element. FIG. 5b shows an opening 52 with a house-like shape (a triangle placed on a rectangle, with the base towards one long edge of the rectangle). FIG. 5c shows a slit-shaped opening 53. FIG. 5d shows a triangular opening 54.

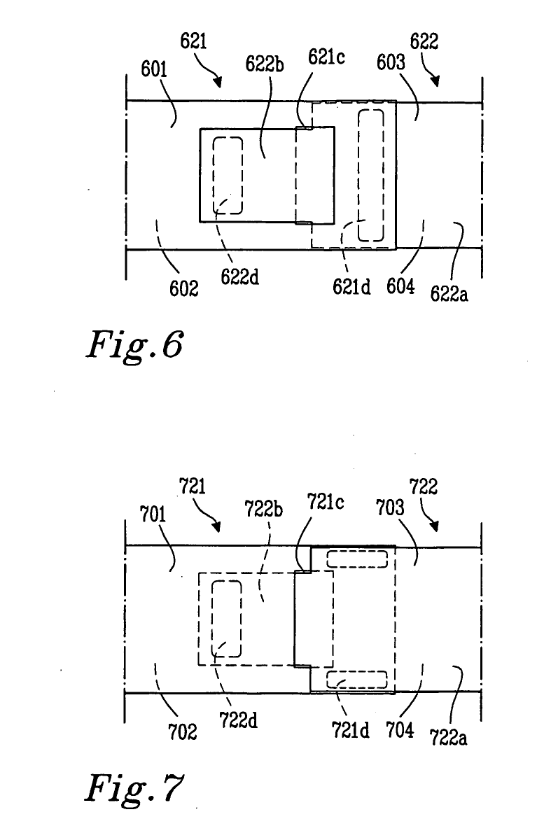

FIGS. 6 and 7 show two different ways to join the receiving portion and the tab element together. The receiving element 621 has an upper face 601 and a lower face 602, and an opening 621c. The tab element 622 has an upper face 603, a lower face 604, a distal portion 622b and a proximal portion 622a. Both the receiving portion 621 and the tab element 622 have fastening means in the form of hook material 621d, 622d placed on the lower face 602, 604.

Upon assembly of the receiving portion 621 and the tab element 622, the distal portion 622b of the tab element 622 is inserted into the opening 621c via the lower face of the receiving portion 621 so that the fastening means 622d of the tab element 622 can fasten to the upper face 601 of the receiving portion 621. At the same time, the fastening means 621d of the receiving portion 621 fasten to the upper face 603 of the tab element 622.

FIG. 7 shows a receiving element 721 with an upper face 701 and a lower face 702, and an opening 721c. The tab element 722 has an upper face 703, a lower face 704, a distal portion 722a and a proximal portion 722b. Both the receiving portion 721 and the tab element 722 have fastening means in the form of hook material 721d, 722d placed on the upper face 701, 703.

Upon assembly of the receiving portion 721 and the tab element 722, the distal portion 722b of the tab element 722 is inserted into the opening 721c via the upper face of the receiving portion 721 so that the fastening means 722d of the tab element 722 can fasten to the lower face 702 of the receiving portion 721. At the same time, the fastening means 721d of the receiving portion 721 fasten to the lower face 704 of the tab element 722.

While the present invention has been described with respect to particular embodiments of the present invention, this is by way of illustration for purposes of disclosure rather than to confine the invention to any specific arrangement as there are various alterations, changes, deviations, eliminations, substitutions, omissions and departures which may be made in the particular embodiment shown and described without departing from the scope of the present invention as defined only by a proper interpretation of the appended claims.

1. Absorbent article comprising:
a fastening device having:
a receiving portion including a proximal portion, a distal portion and an opening located therebetween, and
a tab element including a proximal portion and a distal portion, at least the distal portion of said tab element being shaped so that it can be inserted into the opening of the receiving portion upon closing the article,
wherein both the tab element and the receiving portion include fastening means.

2. Absorbent article according to claim 1 wherein the tab element and the receiving portion have fastening means for attaching to the receiving portion and the tab element, respectively.

3. Absorbent article according to claim 1, wherein the tab element has fastening means in the form of hook material.

4. Absorbent article according to claim 1, wherein the receiving portion has fastening means in the form of hook material.

5. Absorbent article according to claim 4, wherein the receiving portion has fastening means in the form of at least two separate hook material placed adjacent to the opening.

6. Absorbent article according to claim 5, wherein the at least two separate hook materials are located between the proximal portion and the distal portion of the receiving portion.

7. Absorbent article according to claim 6, wherein the at least two separate hook materials extend at least along half of a length of the opening.

8. Absorbent article according to claim 1, wherein the tab element and the receiving portion comprise at least one layer of nonwoven.

9. An absorbent article according to claim 1, wherein the receiving portion is formed from an elastic laminate.

10. An absorbent article according to claim 1, wherein the tab element is formed from an elastic laminate.

11. An absorbent article according to claim 1, wherein the receiving portion comprises elastic zones.

12. An absorbent article according to claim 1, wherein the tab element comprises elastic zones.

13. An absorbent article according to claim 1, wherein the absorbent article is a pant diaper.

14. An absorbent article according to claim 1, wherein the absorbent article is selected from the group consisting of a panty liner, sanitary napkin and light incontinence guard.

15. An absorbent article according to claim 1, wherein the absorbent article is a belt diaper.

16. An absorbent article according to claim 1, wherein the absorbent article is a conventional open diaper.

17. Absorbent article according to claim 3, wherein the receiving portion has fastening means in the form of hook material.

18. Absorbent article according to claim 17, wherein the receiving portion has fastening means in the form of at least two separate hook material placed adjacent to the opening.

19. Absorbent article according to claim 18, wherein the at least two separate hook materials are located between the proximal portion and the distal portion of the receiving portion.

20. Absorbent article according to claim 19, wherein the at least two separate hook materials extend at least along half of a length of the opening.
